(19) United States
(12) Patent Application Publication
(10) Pub. No.: US 2022/0346716 A1
(43) Pub. Date: Nov. 3, 2022

(54) SENSOR COVER

(71) Applicant: Murata Manufacturing Co., Ltd., Kyoto (JP)

(72) Inventors: Hirofumi TSUCHIMOTO, Kyoto (JP); Shinichiro MATSUMOTO, Kyoto (JP); Toru SHIMUTA, Kyoto (JP)

(21) Appl. No.: 17/812,756

(22) Filed: Jul. 15, 2022

Related U.S. Application Data
(63) Continuation of application No. PCT/JP2020/038137, filed on Oct. 8, 2020.

(30) Foreign Application Priority Data
Jan. 30, 2020 (JP) 2020-013271

(51) Int. Cl. A61B 5/00; G01K 13/20; A61B 5/318; G01D 11/24

(52) U.S. Cl. CPC A61B 5/6833; G01K 13/20; A61B 5/318; G01D 11/24

(57) ABSTRACT

A sensor cover includes a cover film having an upper cover film and a lower cover film that are joined to each other at least at respective one sides thereof, and the cover film is shaped like a bag and configured to accommodate a temperature sensor when in use. The sensor cover also includes a self-adhesive member that has adhesiveness and is disposed on an outside surface of the lower cover film. The sensor cover further includes a sensor mounting member that is disposed on an inside surface of the lower cover film and configured to attach the temperature sensor closely to the lower cover film. The sensor cover further includes a sealing member that closes and seals an edge portion of an opening of the cover film.

### CROSS REFERENCE TO RELATED APPLICATION

This is a continuation of International Application No. PCT/JP2020/038137 filed on Oct. 8, 2020 which claims priority from Japanese Patent Application No. 2020-013271 filed on Jan. 30, 2020. The contents of these applications are incorporated herein by reference in their entireties.

### BACKGROUND OF THE DISCLOSURE

### Field of the Disclosure

The present disclosure relates to a sensor cover and more particularly to a sensor cover that encloses a body sensor when the body sensor is adhered to a human body when in use.

### Description of the Related Art

A patch-type body sensor (a patch sensor) is known. The patch-type body sensor is adhered to a human body when in use and acquires biomedical information including, for example, body temperature and electrocardiography signals. When such a patch-type body sensor is used, for example, in a hospital, the body sensor is adhered to a human body using a double-sided adhesive sheet or the like and subsequently covered with a dressing film or a surgical tape adhered thereon, thereby preventing the body sensor, for example, from being stained with an antiseptic solution (for example, Patent Document 1).

Patent Document 1: Japanese Unexamined Patent Application Publication No. 1-201128

### BRIEF SUMMARY OF THE DISCLOSURE

In the case where the above-described patch-type body sensor is used repeatedly (reused), it is typical to do as follows. When in use, the body sensor is adhered to a human body (skin) using a double-sided adhesive sheet that is also adhered to the bottom of the body sensor, and a dressing film being cut into an appropriate size is adhered over the body sensor. After use, the dressing film is peeled off and thrown away, and then the double-sided adhesive sheet is also peeled off, and the body sensor is recovered.

It takes some time and efforts to attach and detach the body sensor and the dressing film or the like, which is not convenient for use. In the case of the body sensor using an exterior material made of a relatively soft material, such as urethane, the exterior material may be damaged when, for example, the dressing film is peeled off.

The present disclosure is made to solve the above problem, and a possible benefit of the present disclosure is to provide a sensor cover for enclosing a body sensor when the body sensor is attached to a human body, the sensor cover being easily replaceable while preventing the body sensor (especially an exterior portion) from being deteriorated or damaged during the replacement.

A sensor cover according to the present disclosure includes a cover film having an upper cover film and a lower cover film that are joined to each other at least at respective one sides thereof, the cover film being shaped like a bag and configured to accommodate a body sensor when in use. The sensor cover also includes a self-adhesive member that has adhesiveness and is disposed on an outside surface of the lower cover film. The sensor cover further includes a sensor mounting member that is disposed on an inside surface of the lower cover film and configured to attach the body sensor closely to the lower cover film. The sensor cover further includes a sealing member that closes and seals an edge portion of an opening of the cover film.

According to the present disclosure, the cover film of the sensor cover has the upper cover film and the lower cover film that are joined to each other at least at respective one sides thereof, and the cover film is shaped like a bag and configured to accommodate the body sensor when in use. This enables the body sensor to be easily put in or taken out the cover film (the space between the upper cover film and the lower cover film). The sensor mounting member is disposed on the inside surface of the lower cover film to adhere the body sensor closely to the lower cover film, which enables the body sensor to be easily attached to the inside surface of the lower cover film (inside the cover film). Moreover, the adhesive material is not adhered to the exterior surface of the body sensor. This can prevent the exterior portion of the body sensor from being deteriorated or damaged when the cover film is detached. In addition, the self-adhesive member having adhesiveness is disposed on the outside surface of the lower cover film, which enables the body sensor to be easily adhered to a human body with the cover film (lower cover film) interposed therebetween. Moreover, the sensor cover has the sealing member that seals the edge portion and closes the opening of the cover film, which enables the cover film to be sealed easily. This can prevent an antiseptic solution or the like from entering.

According to the present disclosure, when the body sensor is attached to a human body when in use, the sensor cover for enclosing the body sensor enables easy replacement of the body sensor while preventing the body sensor (especially the exterior portion) from being deteriorated or damaged during the replacement.

### BRIEF DESCRIPTION OF THE SEVERAL VIEWS OF THE DRAWINGS

FIG. 1 is a plan view illustrating a sensor cover (in use) according to a first embodiment.

FIG. 2 is a cross-sectional view taken along line II-II in FIG. 1.

FIG. 3 is a cross-sectional view illustrating the sensor cover (before use) of the first embodiment.

FIG. 4 is a cross-sectional view illustrating a structure of a patch-type temperature sensor.

FIG. 5 is a plan view illustrating a sensor cover according to a first modification of the first embodiment.

FIG. 6 is a plan view illustrating a sensor cover according to a second modification of the first embodiment.

FIG. 7 is a plan view illustrating a sensor cover (in use) according to a second embodiment.

FIG. 8 is a cross-sectional view taken along line VIII-VIII in FIG. 7.

FIG. 9 is a cross-sectional view illustrating the sensor cover (before use) according to the second embodiment.

FIG. 10 is a plan view illustrating a sensor cover (in use) according to a third embodiment.

FIG. 11 is a cross-sectional view taken along line XI-XI in FIG. 10.

FIG. 12 is a cross-sectional view illustrating the sensor cover (before use) according to the third embodiment.

FIG. 13 is a plan view illustrating a sensor cover (in use) according to a modification example.

FIG. 14 is a cross-sectional view taken along line XIV-XIV in FIG. 13.

FIG. 15 is a cross-sectional view illustrating the sensor cover (before use) according to the modification example.

### DETAILED DESCRIPTION OF THE DISCLOSURE

Several embodiments of the present disclosure will be described in detail with reference to the drawings. Note that the same or equivalent elements will be denoted by the same reference signs in the drawings. In reference to each drawing, duplicated descriptions on the same elements denoted by the same reference signs will be omitted.

### First Embodiment

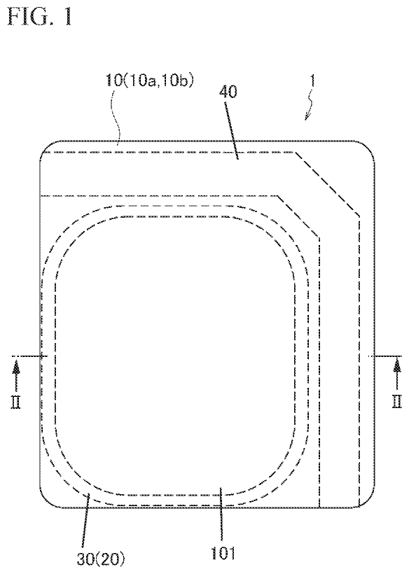

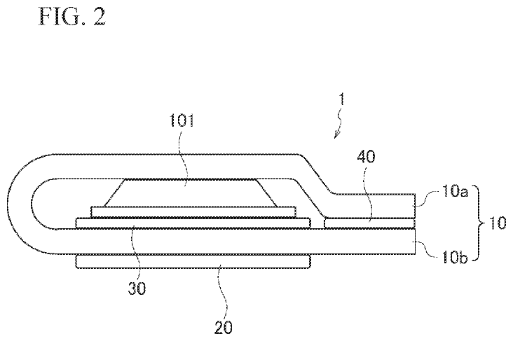

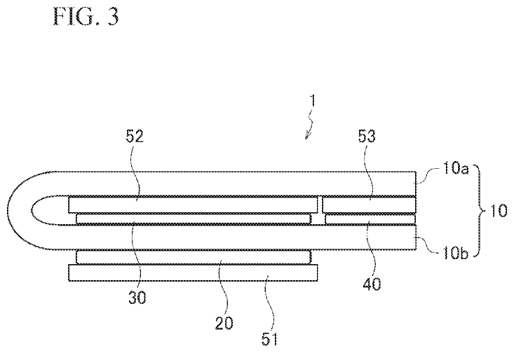

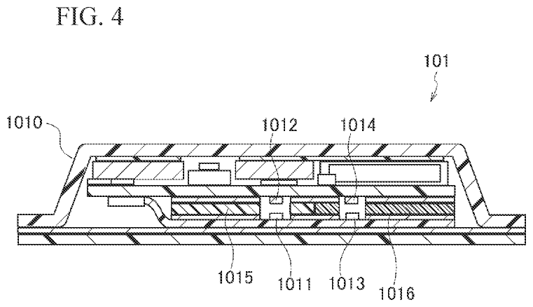

First, a structure of a sensor cover 1 according to a first embodiment will be described with reference to FIGS. 1 to 4. Note that the present disclosure will be described by taking an example in which the present disclosure is applied to a patch-type temperature sensor (deep body thermometer) 101 that is adhered to a human body and measures the deep body temperature of the body. The patch-type temperature sensor corresponds to a body sensor described in Claims. FIG. 1 is a plan view illustrating the sensor cover 1 being in use. FIG. 2 is a cross-sectional view taken along line II-II in FIG. 1. FIG. 3 is a cross-sectional view illustrating the sensor cover 1 before use (for example, in a state after manufacture, on market, or in storage). FIG. 4 is a cross-sectional view illustrating a structure of the patch-type temperature sensor 101.

In the present embodiment, the patch-type temperature sensor 101 is a dual-heat-flux type deep body thermometer equipped with a pair of thermal resistors 1015 and 1016 and two pairs of thermistors 1011, 1012, 1013, and 1014. An example of the dual-heat-flux type deep body thermometer is described in International Publication No. 2019/225532, which can be used as the patch-type temperature sensor 101.

The sensor cover 1 is a cover for enclosing the temperature sensor 101 that is adhered to a body when in use. The sensor cover 1 is a disposable cover. The sensor cover 1 has a function of easy replacement while reducing the likelihood of the patch-type temperature sensor 101 (especially an exterior portion 1010) being deteriorated or damaged during the replacement of the sensor cover 1.

For this purpose, the sensor cover 1 includes, as main components, a cover film 10 constituted of an upper cover film 10a and a lower cover film 10b, a self-adhesive member 20, a sensor mounting member 30, and a sealing member 40.

The upper cover film 10a and the lower cover film 10b of the cover film 10 are joined to each other at least at respective one sides thereof. The cover film 10 is formed as a bag that can accommodate the temperature sensor 101 when in use. In the present embodiment, each of the upper cover film 10a and the lower cover film 10b has a substantially rectangular shape with the corners being rounded off. Two intersecting sides of the upper cover film 10a (for example, the right and bottom sides as in FIG. 1) are joined to corresponding two intersecting sides of the lower cover film 10b, and respective other two sides are open. For example, the cover film 10 may be made of a material having a water proofing property, such as PET.

The self-adhesive member 20 having adhesiveness is disposed on (adhered to) the outside surface of the lower cover film 10b. The self-adhesive member 20 is provided for adhering the sensor cover 1 to a human body while the temperature sensor 101 is enclosed in the sensor cover 1. For example, the self-adhesive member 20 may be a double-sided adhesive member for body use, which is made of silicone or polyethylene (PE) and an acrylic adhesive material. For example, the self-adhesive member 20 has a substantially rectangular shape with the corners being rounded off.

On the other hand, the sensor mounting member 30 is disposed on (adhered to) the inside surface of the lower cover film 10b. The sensor mounting member 30 is provided to adhere the patch-type temperature sensor 101 closely to the lower cover film 10b. The sensor mounting member 30 is disposed so as to oppose the self-adhesive member 20 with the lower cover film 10b being interposed therebetween. The sensor mounting member 30 is an adhesive member having adhesiveness. For example, the sensor mounting member 30 may be a double-sided adhesive member being made of polyethylene (PE) and an acrylic adhesive material. For example, the sensor mounting member 30 has a substantially rectangular shape with the corners being rounded off.

The sealing member 40 is disposed at an edge portion of the opening of the cover film 10 so as to seal the edge portion and close the opening. The sealing member 40 is an adhesive member having adhesiveness. For example, the sealing member 40 may be a double-sided adhesive member made of polyethylene (PE) and an acrylic adhesive material. For example, the sealing member 40 is formed as a sheet shaped substantially like the letter L.

As a result, the cover film 10 is configured, when in use, to have layers of the upper cover film 10a, the temperature sensor 101, the sensor mounting member 30, the lower cover film 10b, and the self-adhesive member 20.

Here, in order to reduce the variation of thermal resistance and thereby improve the accuracy of temperature measurement (i.e., estimation accuracy of deep body temperature), The thermal resistance of the lower cover film 10b may be set to be greater than the thermal resistance of the self-adhesive member 20. The thermal resistance of the lower cover film 10b may be set to be greater than the sum of the thermal resistances of the self-adhesive member 20 and the sensor mounting member 30.

For example, as shown in Table 1, the thickness of the lower cover film 10b may be set to be 300 μm, the thickness of the sensor mounting member 30 to be 50 μm, and the thickness of the self-adhesive member 20 to be 50 μm. In this case, the tolerance of the total thickness of the three layers becomes ±20%, and accordingly, the variation of the thermal resistance can be reduced compared with an example in Table 2, which will be described later. In other words, an increase in the thickness of the lower cover film 10b, which exhibits smaller variation of the thermal resistance, can reduce the variation of the total thermal resistance of the three layers. Accordingly, when the deep body temperature is estimated, the compensation can be made more accurately.

TABLE 1

| | | | | | Minimum | | Maximum | |
|---|---|---|---|---|---|---|---|---|
| | | Tolerance of | Thermal | | thermal | | thermal | |
| Layer | Thickness | thickness | resistance | | resistance | | resistance | |
| Sensor mounting | 50 μm | ±40% | 34° | C./W | 20.4° | C./W | 47.6° | C./W |
| member | | | | | | | | |
| Cover film | 300 μm | ±10% | 240° | C./W | 216° | C./W | 264° | C./W |
| Self-adhesive | 50 μm | ±50% | 50° | C./W | 25° | C./W | 75° | C./W |
| member | | | | | | | | |
| Total | 60 μm | ±20% | 324° | C./W | 261.4° | C./W | 386.6° | C./W |
| | | (equivalent) | | | | | | |

On the other hand, as shown in Table 2, when the thickness of the lower cover film 10b is 15 μm, the thickness of the sensor mounting member 30 is 50 μm, and the thickness of the self-adhesive member 20 is 50 μm, the tolerance of the total thickness of the three layers becomes ±40%, which increases the variation of the thermal resistance compared with the above example in Table 1.

TABLE 2

| | | | | | Minimum | | Maximum | |
|---|---|---|---|---|---|---|---|---|
| | | Tolerance of | Thermal | | thermal | | thermal | |
| Layer | Thickness | thickness | resistance | | resistance | | resistance | |
| Sensor mounting | 50 μm | ±40% | 34° | C./W | 20.4° | C./W | 47.6° | C./W |
| member | | | | | | | | |
| Cover film | 15 μm | ±10% | 12° | C./W | 10.8° | C./W | 13.2° | C./W |
| Self-adhesive | 50 μm | ±50% | 50° | C./W | 25° | C./W | 75° | C./W |
| member | | | | | | | | |
| Total | 20 μm | ±40% | 96° | C./W | 56.2° | C./W | 135.8° | C./W |
| | | (equivalent) | | | | | | |

When the above variation of the tolerance of the thickness (i.e., the variation of the thermal resistance) is taken into account, the thickness of the lower cover film 10b may be selected in the range of 30 to 2000 μm in the case where the thermal resistance of the lower cover film 10b is greater than those of the sensor mounting member 30 and the self-adhesive member 20 (double-sided adhesive member).

Next, the configuration of the sensor cover 1 before use (in a state, for example, after manufacture, on market, or in storage) will be described with reference to FIG. 3. FIG. 3 is a cross-sectional view of the sensor cover 1 before use.

Before use, the sensor cover 1 has a first separator 51, a second separator 52, and a third separator 53. The first separator 51 is releasably adhered to the above-described self-adhesive member 20 (double-sided adhesive member for body use) so as to cover the self-adhesive member 20. The second separator 52 is releasably adhered to the sensor mounting member 30 (double-sided adhesive member) so as to cover the sensor mounting member 30. The third separator 53 is releasably adhered to the sealing member 40 (double-sided adhesive member) so as to cover the sealing member 40. Note that in place of the second separator 52 and the third separator 53, for example, a silicone-based release agent may be applied to the inside surface of the upper cover film 10a.

The first separator 51, the second separator 52, and the third separator 53 are members that are adhered to adhesive surfaces of the sensor cover 1 (i.e., surfaces of the self-adhesive member 20, sensor mounting member 30, and the sealing member 40) when the sensor cover 1 is not in use. The first separator 51, the second separator 52, and the third separator 53 are peeled off the adhesive surfaces of the sensor cover 1 (i.e., surfaces of the self-adhesive member 20, sensor mounting member 30, and the sealing member 40) when the sensor cover 1 is used.

When the patch-type temperature sensor 101 is used, the first separator 51 is first peeled off, and the temperature sensor 101 is adhered to the sensor mounting member 30 (double-sided adhesive member). Next, the second separator 52 is peeled off, and the opening (between the upper cover film 10a and the lower cover film 10b) of the cover film 10 is closed and sealed using the sealing member 40. Consequently, the third separator 53 is peeled off, and the temperature sensor 101 enclosed in the cover film 10 (sensor cover 1) is adhered to a human body (skin).

After the temperature of the body (deep body temperature) is measured, in other words, after the patch-type temperature sensor 101 is used, the temperature sensor 101 enclosed in the cover film 10 (sensor cover 1) is detached from the body (skin). Next, the cover film 10 is opened, and the temperature sensor 101 is detached from the sensor mounting member 30 (double-sided adhesive member). Thus, the temperature sensor 101 is recovered. The sensor cover 1 is thrown away after use.

According to the present embodiment, as described above in detail, the cover film 10 has the upper cover film 10a and the lower cover film 10b that are joined to each other at least at respective one sides thereof, and the cover film 10 is formed as a bag that can accommodate the temperature sensor 101 when in use. This enables the temperature sensor 101 to be put in or taken out the cover film 10 easily. In addition, the sensor mounting member 30 is disposed on the inside surface of the lower cover film 10b to adhere the temperature sensor 101 closely to the lower cover film 10b, which enables the temperature sensor 101 to be easily attached to the inside surface of the lower cover film 10b (inside the cover film 10). The adhesive material is not adhered to the exterior surface of the temperature sensor 101. This prevents the exterior portion 1010 of the temperature sensor 101 from being deteriorated or damaged when the cover film 10 is detached. In addition, the self-adhesive member 20 having adhesiveness is disposed on the outside surface of the lower cover film 10b, which enables the temperature sensor 101 to be easily adhered to a human body with the cover film 10 (lower cover film 10b) interposed therebetween. Moreover, the sensor cover has the sealing member 40 that seals the edge portion and closes the opening of the cover film 10, which enables the cover film 10 to be sealed easily. This can prevent an antiseptic solution or the like from entering.

Thus, according to the present embodiment, the temperature sensor 101 can be replaced easily while reducing the likelihood of the temperature sensor 101 (especially the exterior portion 1010) being deteriorated or damaged during the replacement of the sensor cover 1.

According to the present embodiment, the sensor mounting member 30 is the adhesive member having adhesiveness, which enables the temperature sensor 101 to be mounted or detached easily.

According to the present embodiment, the upper cover film 10a and the lower cover film 10b are joined to each other at respective two intersecting sides, and the sealing member 40 is the adhesive member having adhesiveness. This enables both easy mounting (easy positioning) of the temperature sensor 101 and easy sealing and opening.

According to the present embodiment, the thermal resistance of the lower cover film 10b is set to be greater than the sum of the thermal resistances of the self-adhesive member 20 and the sensor mounting member 30, which can reduce the variation of the total thermal resistance and can improve, for example, the estimation accuracy of deep body temperature.

According to the present embodiment, the sensor cover 1 before use has the first separator 51 that is releasably adhered to the self-adhesive member 20, the second separator 52 that is releasably adhered to the sensor mounting member 30, and the third separator 53 that is releasably adhered to the sealing member 40. These separators can store the sensor cover 1 while protecting the adhesive surfaces of the self-adhesive member 20, the sensor mounting member 30, and the sealing member 40. The first separator 51, the second separator 52, and the third separator 53 are peeled off when in use. This makes it easier to adhere the temperature sensor 101 to the sensor cover 1, to seal the sensor cover 1, and also to adhere the sensor cover 1 (temperature sensor 101) to a human body.

### First Modification

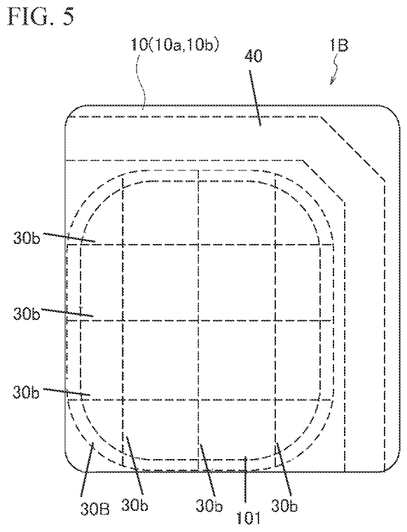

Next, a sensor cover 1B according to a first modification of the first embodiment will be described with reference to FIG. 5. FIG. 5 is a plan view of the sensor cover 1B.

The sensor cover 1B is different from the above-described sensor cover 1 in that the sensor cover 1B includes a sensor mounting member 30B in place of the sensor mounting member 30. The sensor mounting member 30B has ventilation passages 30b formed so as to at least connect the central portion of the sensor mounting member 30B to the outer edge thereof as viewed in plan. More specifically, slits 30b serving as the ventilation passages 30b are formed in the sensor mounting member 30B so as to form a grid. The formation of the slits 30b is not limited to the shape illustrated in FIG. 5 but may be shaped like lines radially extending from the center. In place of forming the slits 30b, the sensor mounting member 30B may be disposed so as to form dotted shapes in order to form the ventilation passages.

In the present modification, the ventilation passages (slits) 30b are formed in the sensor mounting member 30B, which can prevent air pockets from forming between the temperature sensor 101 and the sensor mounting member 30B when the temperature sensor 101 is adhered thereto. Note that in this case, a layer of air remains between the temperature sensor 101 and the sensor mounting member 30B, but the amount of air remaining can be determined quantitatively in advance so that the air can be taken into calculation, thereby preventing the deterioration of the measurement accuracy.

Note that the other configurations are the same as or similar to those of the sensor cover 1 of the first embodiment above, and detailed descriptions thereof are not repeated here.

### Second Modification

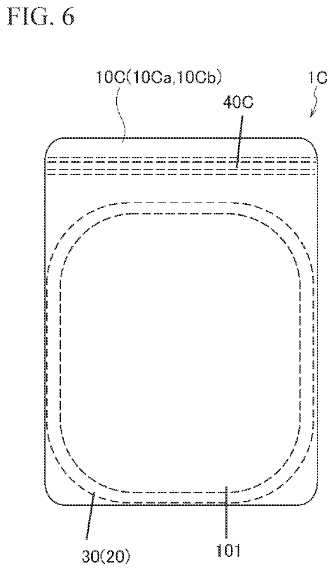

In the above embodiment, the upper cover film 10a and the lower cover film 10b are joined at respective two sides thereof. The upper cover film 10a and the lower cover film 10b, however, may be joined at respective three sides as illustrated in FIG. 6. A sensor cover 1C according to a second modification of the first embodiment will be described with reference to FIG. 6. FIG. 6 is a plan view of the sensor cover 1C.

The sensor cover 1C is different from the above-described sensor cover 1 in that the sensor cover 1C has an upper cover film 10Ca and a lower cover film 10Cb that are joined to each other at respective three sides thereof (in other words, the sensor cover 1C can open at one side). Another difference from the above sensor cover 1 is that a sealing member 40C is formed as an engagement member of a mutual engagement type (fastener).

According to the present modification, the upper cover film 10Ca and the lower cover film 10Cb are joined at respective three sides, and the sealing member 40C is formed as the engagement member of the mutual engagement type (fastener), which enables the cover film 10C to be sealed and opened more easily.

Note that the other configurations are the same as or similar to those of the sensor cover 1 of the first embodiment above, and detailed descriptions thereof are not repeated here.

### Second Embodiment

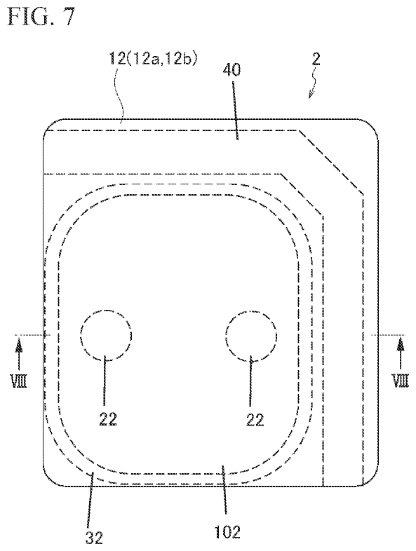

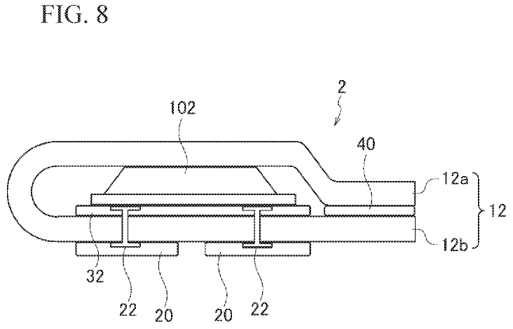

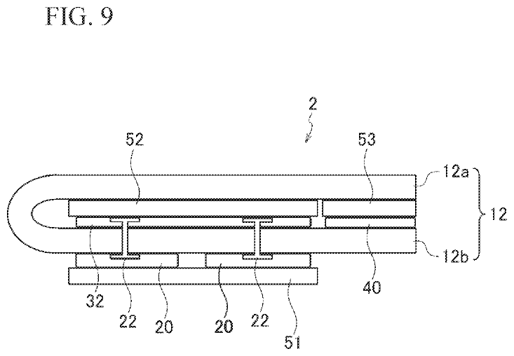

In the first embodiment above, the present disclosure is described using an example in which the present disclosure is applied to the patch-type temperature sensor 101. The present disclosure, however, can be applied also to a patch-type electrocardiography sensor. Next, the present disclosure will be described with reference to FIGS. 7 to 9 by taking an example in which the present disclosure is applied to a patch-type electrocardiography sensor 102 that is adhered to a human body and acquires electrocardiography signals. The patch-type electrocardiography sensor 102 corresponds to the body sensor described in Claims. FIG. 7 is a plan view illustrating a sensor cover 2 (in use) according to a second embodiment. FIG. 8 is a cross-sectional view taken along line VIII-VIII in FIG. 7. FIG. 9 is a cross-sectional view of the sensor cover 2 (before use).

When in use, a cover film 12 of the sensor cover 2 to be adhered to a human body accommodates the patch-type electrocardiography sensor 102 that acquires electrocardiography signals of the body. Conductive members 22 having electric conductivity are disposed so as to pierce a lower cover film 12b and a sensor mounting member 32 in the thickness direction thereof and so as to oppose electrocardiography electrodes of the patch-type electrocardiography sensor 102 when the patch-type electrocardiography sensor 102 is in use and is attached closely to the lower cover film 12b. The lower cover film 12b may be made of an anisotropic material that exhibits electroconductivity only in the thickness direction instead of having the conductive members 22.

According to the present embodiment, the conductive members 22 are formed in the lower cover film 12b and the sensor mounting member 32 so as to pierce the lower cover film 12b and the sensor mounting member 32 and so as to oppose the electrocardiography electrodes of the electrocardiography sensor 102, which enables the electrocardiography sensor 102 to detect electrocardiography signals sensitively.

Note that the other configurations are the same as or similar to those of the sensor cover 1 of the first embodiment above, and detailed descriptions thereof are not repeated here.

### Third Embodiment

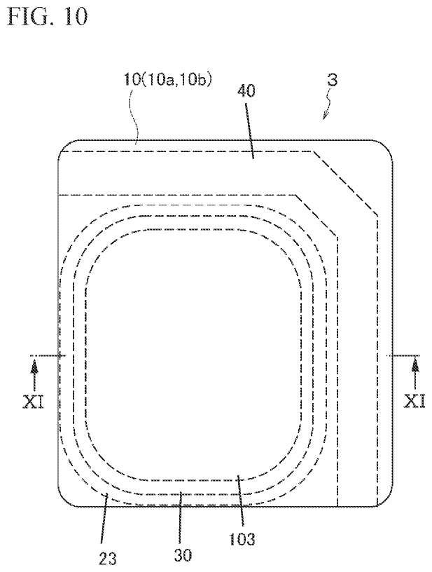

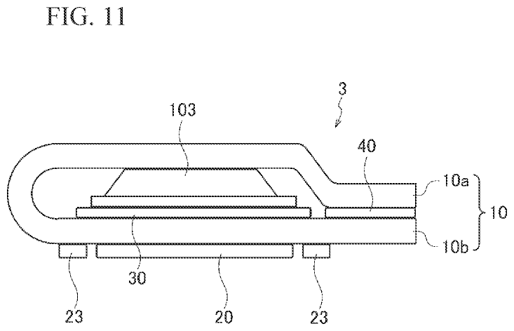

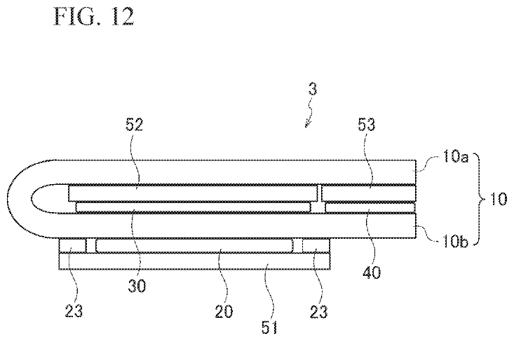

In the first embodiment above, the present disclosure is described using an example in which the present disclosure is applied to the patch-type temperature sensor 101. The present disclosure, however, can be applied also to a patch-type acceleration sensor. Next, the present disclosure will be described with reference to FIGS. 10 to 12 by taking an example in which the present disclosure is applied to a patch-type acceleration sensor 103 that is adhered to a human body and detects, for example, the position and motion of the body. The patch-type acceleration sensor 103 also corresponds to the body sensor described in Claims. FIG. 10 is a plan view illustrating a sensor cover 3 (in use) according to a third embodiment. FIG. 11 is a cross-sectional view taken along line XI-XI in FIG. 10. FIG. 12 is a cross-sectional view of the sensor cover 3 (before use).

When in use, a cover film 10 of the sensor cover 3 to be adhered to a human body accommodates the patch-type acceleration sensor 103 that detects, for example, the position and motion of the body. The self-adhesive member 20 is configured such that the peripheral portion is more rigid than the central portion. More specifically, a rectangular frame 23 is disposed at the periphery of the self-adhesive member 20 (in other words, disposed so as to surround the self-adhesive member 20), and the frame 23 is more rigid than the self-adhesive member 20.

According to the present embodiment, the rectangular frame 23 that is more rigid than the self-adhesive member 20 is disposed at the periphery of the self-adhesive member 20, in other words, the peripheral portion is made more rigid than the central portion. This enables the acceleration sensor 103 to be fixed more rigidly (in other words, to be prevented from moving laterally), which can improve the detection accuracy of the acceleration. Another frame may be provided at the periphery of the sensor mounting member 30 in addition to the frame at the periphery of the self-adhesive member 20.

Note that the other configurations are the same as or similar to those of the sensor cover 1 of the first embodiment above, and detailed descriptions thereof are not repeated here.

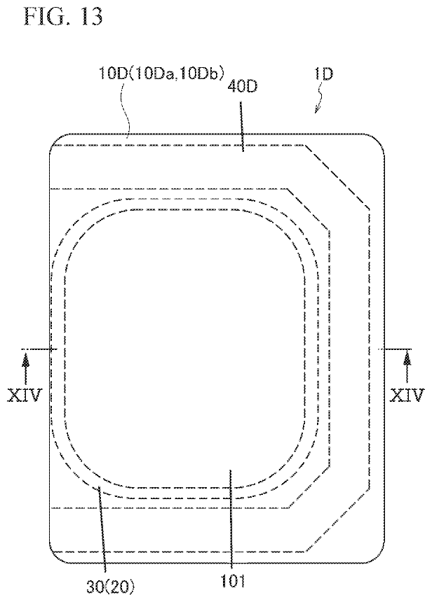

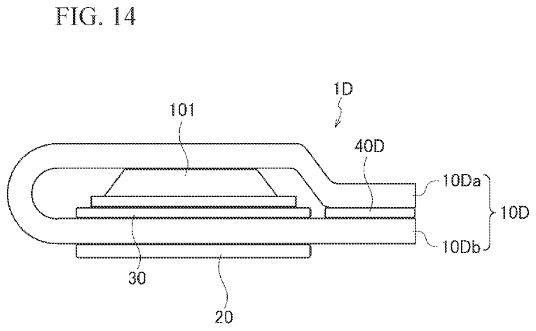

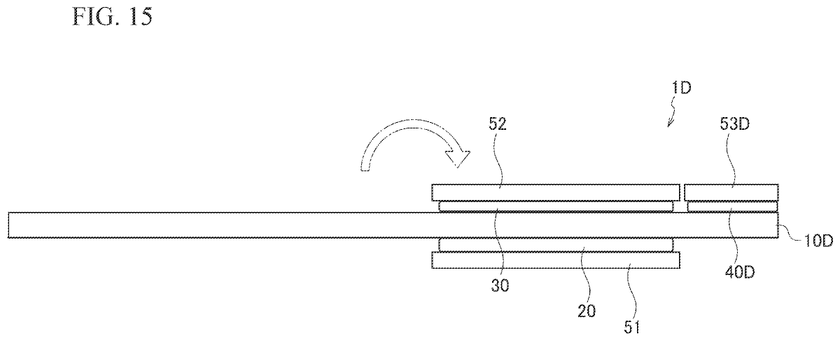

Embodiments of the present disclosure have been described. The present disclosure, however, is not limited to the above embodiments but may be subjected to various alterations. For example, in the above embodiment, the upper cover film 10a and the lower cover film 10b are joined at respective two or three sides thereof. The upper cover film 10a and the lower cover film 10b, however, may be joined at respective one sides as illustrated in FIGS. 13 to 15. In such a case, a cover film 10D is folded, when in use, at one side thereof (see FIG. 15) and the other three sides are sealed using double-sided adhesive members (double-sided adhesive tapes) 40D or the like. FIG. 13 is a plan view illustrating a sensor cover 1D (in use) according to a modification example, and FIG. 14 is a cross-sectional view taken along line XIV-XIV in FIG. 13. FIG. 15 is a cross-sectional view illustrating the sensor cover 1D (before use) according to the modification example. With this configuration, the sensor cover 1D opens at three sides, which can not only make it easier to attach and detach the body sensor but also improve the cost efficiency in manufacturing the sensor cover 1D because a step of folding the sensor cover 1D is not necessary.

In the above embodiments, the present disclosure has been described using an example in which the present disclosure is applied to the temperature sensor 101, the electrocardiography sensor 102, and the acceleration sensor 103, which are of the patch-type. The present disclosure, however, can be applied to patch-type body sensors other than the temperature sensor 101, the electrocardiography sensor 102, and the acceleration sensor 103, which are of the patch-type.

In addition, the double-sided adhesive member is used for the sensor mounting member 30 in the above embodiments. It is sufficient, however, that the sensor mounting member 30 can serve to attach the bottom surface of a body sensor closely to the lower cover film 10b. For example, the sensor cover may be configured to fix the four corners of the body sensor, or the sensor cover may be configured to have a pocket of a size corresponding substantially to the thickness of the body sensor.

1, 1B, 1C, 1D, 2, 3 sensor cover

10, 10C, 10D, 12 cover film

10a, 10Ca, 10Da, 12a upper cover film

10b, 10Cb, 10Db, 12b lower cover film

20 self-adhesive member (double-sided adhesive member for body use)

22 conductive member

23 frame

30, 30B, 32 sensor mounting member (double-sided adhesive member)

30b slit (ventilation passage)

40, 40D sealing member (double-sided adhesive member)

40C sealing member (engagement member)

51, 52, 53, 53D separator

101 patch-type temperature sensor

102 patch-type electrocardiography sensor

103 patch-type acceleration sensor

1. A sensor cover, comprising:
a cover film having an upper cover film and a lower cover film, the upper cover film and the lower cover film being joined to each other at least at respective one sides thereof, the cover film being shaped like a bag and configured to accommodate a body sensor when in use;
a self-adhesive member having adhesiveness and disposed on an outside surface of the lower cover film;
a sensor mounting member disposed on an inside surface of the lower cover film and configured to attach the body sensor closely to the lower cover film; and
a sealing member closing and sealing an edge portion of an opening of the cover film.

2. The sensor cover according to claim 1, wherein
the sensor mounting member is an adhesive member having adhesiveness.

3. The sensor cover according to claim 2, wherein
the sensor mounting member has a ventilation passage provided so as to at least connect a central portion of the sensor mounting member to an outer edge thereof as viewed in plan.

4. The sensor cover according to claim 1, wherein
the upper cover film and the lower cover film are shaped substantially like a rectangle and are joined to each other at respective two intersecting sides thereof, and
the sealing member is an adhesive member having adhesiveness.

5. The sensor cover according to claim 1, wherein
the upper cover film and the lower cover film are shaped substantially like a rectangle and are joined to each other at respective three sides thereof, and
the sealing member is an engagement member of a mutual engagement type.

6. The sensor cover according to claim 1, wherein
when in use, the cover film is adhered to a human body and accommodates a patch-type temperature sensor configured to measure body temperature of the human body, and
a thermal resistance of the lower cover film is set to be greater than a thermal resistance of the self-adhesive member.

7. The sensor cover according to claim 1, wherein
when in use, the cover film is adhered to a human body and accommodates a patch-type temperature sensor configured to measure body temperature of the human body, and
a thermal resistance of the lower cover film is set to be greater than a sum of a thermal resistance of the self-adhesive member and a thermal resistance of the sensor mounting member.

8. The sensor cover according to claim 1, wherein
when in use, the cover film is adhered to a human body and accommodates a patch-type electrocardiography sensor configured to acquire electrocardiography signals of the human body, and
a conductive member having electric conductivity is disposed in the lower cover film and in the sensor mounting member at such a position that the conductive member opposes an electrocardiography electrode of the electrocardiography sensor when the electrocardiography sensor is attached closely to the lower cover film when in use.

9. The sensor cover according to claim 1, wherein
when in use, the cover film is adhered to a human body and accommodates a patch-type acceleration sensor configured to detect position and motion of the human body, and
the sensor mounting member and/or the self-adhesive member is configured such that a peripheral portion thereof is more rigid than a central portion thereof.

10. A sensor cover, comprising:
a cover film having an upper cover film and a lower cover film, the upper cover film and the lower cover film being joined to each other at least at respective one sides thereof, the cover film being shaped like a bag and configured to accommodate a body sensor when in use;
a self-adhesive member having adhesiveness and disposed on an outside surface of the lower cover film;
a sensor mounting member disposed on an inside surface of the lower cover film and configured to attach the body sensor closely to the lower cover film, the sensor mounting member being comprised of an adhesive member having adhesiveness;
a sealing member closing and sealing an edge portion of an opening of the cover film and being comprised of an adhesive member having adhesiveness;
a first separator releasably adhered to the self-adhesive member;
a second separator releasably adhered to the sensor mounting member; and
a third separator releasably adhered to the sealing member.

11. The sensor cover according to claim 2, wherein
the upper cover film and the lower cover film are shaped substantially like a rectangle and are joined to each other at respective two intersecting sides thereof, and
the sealing member is an adhesive member having adhesiveness.

12. The sensor cover according to claim 3, wherein
the upper cover film and the lower cover film are shaped substantially like a rectangle and are joined to each other at respective two intersecting sides thereof, and
the sealing member is an adhesive member having adhesiveness.

13. The sensor cover according to claim 2, wherein
the upper cover film and the lower cover film are shaped substantially like a rectangle and are joined to each other at respective three sides thereof, and
the sealing member is an engagement member of a mutual engagement type.

14. The sensor cover according to claim 3, wherein
the upper cover film and the lower cover film are shaped substantially like a rectangle and are joined to each other at respective three sides thereof, and
the sealing member is an engagement member of a mutual engagement type.

15. The sensor cover according to claim 2, wherein
when in use, the cover film is adhered to a human body and accommodates a patch-type temperature sensor configured to measure body temperature of the human body, and
a thermal resistance of the lower cover film is set to be greater than a thermal resistance of the self-adhesive member.

16. The sensor cover according to claim 3, wherein
when in use, the cover film is adhered to a human body and accommodates a patch-type temperature sensor configured to measure body temperature of the human body, and
a thermal resistance of the lower cover film is set to be greater than a thermal resistance of the self-adhesive member.

17. The sensor cover according to claim 4, wherein
when in use, the cover film is adhered to a human body and accommodates a patch-type temperature sensor configured to measure body temperature of the human body, and
a thermal resistance of the lower cover film is set to be greater than a thermal resistance of the self-adhesive member.

18. The sensor cover according to claim 5, wherein
when in use, the cover film is adhered to a human body and accommodates a patch-type temperature sensor configured to measure body temperature of the human body, and
a thermal resistance of the lower cover film is set to be greater than a thermal resistance of the self-adhesive member.

19. The sensor cover according to claim 2, wherein
when in use, the cover film is adhered to a human body and accommodates a patch-type temperature sensor configured to measure body temperature of the human body, and
a thermal resistance of the lower cover film is set to be greater than a sum of a thermal resistance of the self-adhesive member and a thermal resistance of the sensor mounting member.

20. The sensor cover according to claim 3, wherein
when in use, the cover film is adhered to a human body and accommodates a patch-type temperature sensor configured to measure body temperature of the human body, and
a thermal resistance of the lower cover film is set to be greater than a sum of a thermal resistance of the self-adhesive member and a thermal resistance of the sensor mounting member.
